(19) United States
(12) Patent Application Publication
(10) Pub. No.: US 2025/0339354 A1
(43) Pub. Date: Nov. 6, 2025

(54) CONCENTRATED CONDITIONING COMPOSITIONS

(71) Applicant: Conopco, Inc., d/b/a UNILEVER, Englewood Cliffs, NJ (US)

(72) Inventors: Christine BECKWITH, Meriden, CT (US); Karoline Pugnaloni DA SILVA, Bridgeport, CT (US); Leonardus Cornelis Maria van GORKOM, Naugatuck, CT (US)

(73) Assignee: Conopco, Inc., d/b/a UNILEVER, Englewood Cliffs, NJ (US)

(21) Appl. No.: 18/867,060

(22) PCT Filed: Apr. 24, 2023
(86) PCT No.: PCT/EP2023/060587

§ 371 Date: Nov. 19, 2024

Related U.S. Application Data
(60) Provisional application No. 63/346,351, filed on May 27, 2022.

(30) Foreign Application Priority Data
Jul. 15, 2022 (EP) 22185205.6

(51) Int. Cl. A61K 8/34; A61Q 5/00; A61Q 5/12

(52) U.S. Cl. CPC A61K 8/342; A61Q 5/002; A61Q 5/12; A61K 2800/48; A61K 2800/5426

(57) ABSTRACT

A concentrated conditioning composition comprises: 6 to 10% by weight of a fatty alcohol; 1.0 to 10.0% by weight of a conditioning agent comprising a cationic surfactant, cationic polymers, or a combination thereof; a humectant, wherein the humectant comprises propylene glycol, butylene glycol, dipropylene glycol, glycerin, triethylene glycol, erythritol, capryl glycol, hyaluronic acid, polypropylene glycol-7 proypyl heptyl ether, or a combination thereof; a thickening agent; and 60 to 90% by weight of water. The concentrated conditioning composition has a level of concentration of 2.5, preferably wherein the level of concentration is 2.0, as compared to a base, non-concentrated conditioning composition.

### FIELD OF THE INVENTION

Disclosed herein are concentrated conditioning compositions. The concentrated conditioning compositions comprise 6 to 10% by weight of a fatty alcohol; 1.0 to 10.0% by weight of a conditioning agent comprising a cationic surfactant, cationic polymer, or a combination thereof; and 60 to 90% by weight of water.

### BACKGROUND OF THE INVENTION

Liquid based cleansing compositions, such as shampoos, body washes, conditioners, and hand washes are common and enjoyed by many consumers. Such compositions typically have water as the predominant ingredient, and they are often sold in plastic bottles or tubes. The compositions are conventionally formulated to have a viscosity that is customary for consumer use and easy for evacuation from the package in which they are sold.

It is often publicized that the world's oceans will soon have more plastic than fish. Given environmental concerns and the desire for consumers and conscientious companies to do more for the planet, there is a strong desire to use less plastic when selling products, including consumer products. In view of this, efforts have been made to sell product in concentrate form, and therefore, ship product that comprises less water. The difficulty with concentrates is consumers often do not like adding additional water to the concentrate and further work, like stirring, to convert the concentrate into an end usable product. As to the hydrated product, common complaints include that the product is not homogeneous after adding water and/or of undesirable viscosity.

Efforts have been disclosed for making wash compositions. In U.S. Patent Publication No. 2019/031258 A1, rheo-fluidifying concentrated foaming compositions are described.

Other efforts have been disclosed for making wash compositions. In U.S. Patent Publication No. 2018/098923 A1, personal care compositions substantially free of sulfated surfactants are described.

Still other efforts have been disclosed for making wash compositions. In U.S. Patent Publication No. 2019/282480 A1, self-thickening cleansing compositions with N-acyl acidic amino acids or salts thereof and an amphoteric surfactant are described.

U.S. Pat. No. 9,308,398 B2 discloses a multiple product system regimen for providing improved benefits to hair.

It is of increasing interest to develop a concentrate that is easy to pour and that does not need to be hydrated, resulting in a consumer product that is ready to use immediately and has very desirable characteristics, including viscosity. It is also desirable to develop a concentrate easy to use in a smaller package to reduce plastic waste.

### SUMMARY OF THE INVENTION

Disclosed in various aspects are concentrated conditioning compositions.

A concentrated conditioning composition comprises: 6 to 10% by weight of a fatty alcohol based on the total weight of the concentrated conditioning composition; 1.0 to 10.0% by weight of a conditioning agent comprising a cationic surfactant, cationic polymer, or a combination thereof based on the total weight of the concentrated conditioning composition; a humectant, wherein the humectant comprises propylene glycol, butylene glycol, dipropylene glycol, glycerin, triethylene glycol, erythritol, capryl glycol, hyaluronic acid, polypropylene glycol-7 proypyl heptyl ether, or a combination thereof; a thickening agent; and 60 to 90% by weight of water based on the total weight of the concentrated conditioning composition.

These and other features and characteristics are more particularly described below.

### DETAILED DESCRIPTION OF THE INVENTION

Disclosed herein is a concentrated conditioning composition. The concentrated conditioning composition comprises a fatty alcohol, a conditioning agent, and water. The concentrated conditioning composition can be used directly from the container in which it is packaged without adding hydration. Not needing to add hydration saves the end user or consumer the added step of having to re-hydrate the product with the correct amount of hydration and mixing sufficiently to ensure homogeneity. The present conditioning composition is concentrated at a level of up to 2.5 times as compared to a base composition that is not concentrated. For example, the conditioning composition can be concentrated at a level of 2.0 times as compared to a base composition that is not concentrated. Conditioners containing high levels of surfactant (i.e., conditioning agent) are typically more viscous, due to the high levels of solid present. Such high levels of conditioning agent generally result in more viscous compositions that don't have a flowable rheology or are easily expelled from their container, and are more difficult to process, especially in large scale batches. Surprisingly, the concentrated conditioning composition disclosed herein has a viscosity that allows it to be processed in large scale batches, has a flowable rheology, and can be packaged in the same type of containers used for non-concentrated compositions. It was unexpectedly found that parity performance with current non-concentrated conditioners can be achieved with the present concentrated conditioning composition.

Since the concentrated conditioning composition does not have to be re-hydrated, a smaller dose of the concentrated conditioning composition can be used by the end user. For example, a 2.5 times concentrated conditioning composition would have a 2.5 times smaller dose, a 2.0 times concentrated conditioning composition would have a 2.0 times smaller dose, a 1.5 times concentrated conditioning composition would have a 1.5 times smaller dose and so on.

The concentrated conditioning composition can contain 6 to 10% by weight of a fatty alcohol based on the total weight of the concentrated conditioning composition. The fatty alcohol can comprise a C₆ to C₂₂ alcohol, preferably the fatty alcohol comprises a C₈ to C₂₀ alcohol, more preferably the fatty alcohol comprises a C₁₀ to C₁₈ alcohol, and even more preferably the fatty alcohol comprises a C₁₆ to C₁₈ alcohol. The fatty alcohol can comprise lauryl alcohol, myristyl alcohol, cetyl alcohol, stearyl alcohol, oleyl alcohol, or a combination thereof.

The concentrated conditioning composition can contain 1.0 to 10.0% by weight of a conditioning agent based on the total weight of the concentrated conditioning composition. For example, the concentrated conditioning agent can contain 1.05 to 6.5% by weight of the conditioning agent based on the total weight of the concentrated conditioning composition. The conditioning agent can comprise a cationic surfactant, cationic polymer, or a combination thereof.

The cationic surfactant conditioning agent can comprise behentrimonium chloride, stearamidopropyl dimethylamine, cetyl trimethyl ammonium chloride, stearyl trimethyl ammonium chloride, hydrogenated tallow alkyl trimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, N-(stearoyl colamino formyl methy) pyridinium chloride, or a combination thereof.

One class of cationic surfactant conditioning agent includes heterocyclic ammonium salts such as cetyl or stearyl pyridinium chloride, alkyl amidoethyl pyrrylinodium methyl sulfate, lapyrium chloride, or a combination thereof.

Tetra alkyl ammonium salts are another useful class of cationic surfactant conditioning agents. Examples include cetyl or stearyl trimethyl ammonium chloride or bromide, hydrogenated palm or tallow trimethylammonium halides, behenyl trimethyl ammonium halides or methyl sulfates, decyl isononyl dimethyl ammonium halides, ditallow (or distearyl) dimethyl ammonium halides, behenyl dimethyl ammonium chloride, or a combination thereof.

Still other types of cationic surfactant conditioning agent that can be used are the various ethoxylated quaternary amines and ester quats. Examples include PEG-5 stearyl ammonium lactate (e.g., Genamin KSL manufactured by Clariant), PEG-2 coco ammonium chloride, PEG-15 hydrogenated tallow ammonium chloride, PEG 15 stearyl ammonium chloride, dipalmitoyl ethyl methyl ammonium chloride, dipalmitoyl hydroxyethyl methyl sulfate, stearyl amidopropyl dimethylamine lactate, or a combination thereof.

Even other useful cationic surfactant conditioning agents include quaternized hydrolysates of silk, wheat, and keratin proteins, or a combination thereof. Oat peptide is another useful additive in the concentrated conditioning compositions.

The cationic surfactant conditioning agent can be present in the concentrated conditioning composition in an amount of 1.0 to 5.0% by weight, for example, 1.25 to 3.5% by weight, for example, 1.4 to 2.0% by weight, for example, 1.5 to 3.0% by weight, for example, 1.75 to 2.5% by weight based on the total weight of the concentrated conditioning compositions, including any and all ranges and values subsumed therein.

The cationic polymer conditioning agent can comprise copolymers of 1-vinyl-2-pyrrolidone and 1-vinyl-3-methylimidazolium salt; copolymers of 1-vinyl-2-pyrrolidone and dimethylaminoethyl methacrylate; cationic diallyl quaternary ammonium-containing polymers, or a combination thereof. The cationic polymer conditioning agent can be present in the concentrated conditioning composition in an amount of 0.1 to 5% by weight, for example, 0.25 to 4% by weight, for example, 0.5 to 3% by weight, for example, 1.0 to 2.5% by weight, for example, 0.1 to 1.0% by weight, based on the total weight of the concentrated conditioning composition, including any and all ranges and values subsumed therein.

The cationic diallyl quaternary ammonium-containing polymers can comprise dimethyldiallylammonium chloride homopolymer and copolymers of acrylamide and dimethyldiallylammonium chloride. The cationic diallyl quaternary ammonium-containing polymers can be present in an amount of 0.1 to 5% by weight, for example, 0.25 to 4% by weight, for example, 0.5 to 3% by weight, for example, 0.1 to 2.5% by weight, for example, 0.1 to 1.0% by weight based on the total weight of the concentrated conditioning composition, including any and all ranges and values subsumed therein.

The conditioning agent can additionally optionally comprise isohexadecane, a silicone, or a combination thereof. When present, the silicone conditioning agent can comprise dimethicone, amodimethicone, cyclomethicone, dimethiconol and dimethiconol/silsesquioxane copolymeror a combination thereof. The conditioning agent can be present in the concentrated conditioning composition in an amount of 0.25 to 5.0% by weight, for example, 0.29 to 3.75% by weight, for example, 0.35 to 3.7% by weight, for example, 0.29 to 2.8% by weight, for example, 0.37 to 3.5% by weight, based on the total weight of the concentrated conditioning composition, including any and all ranges and values subsumed therein. A combination of dimethicone and amodimethicone can be used in the concentrated conditioning composition. When such a combination is present, the level of dimethicone present in the concentrated conditioning composition can be 4 to 6%, for example, 4.6% of the concentrated conditioning composition, based on the total weight of the concentrated conditioning composition, including any and all ranges and values subsumed therein and the level of amodimethicone can be 0.1 to 1.0% by weight of the concentrated conditioning composition, for example, 0.5% by weight of the concentrated conditioning composition, based on the total weight of the concentrated conditioning composition, including any and all ranges and values subsumed therein.

The concentrated conditioning composition can comprise water in an amount of 30 to 95% by weight, for example, 60 to 90% by weight, for example, 40 to 78% by weight, based on the total weight of the concentrated conditioning composition, including any and all ranges and values subsumed therein.

Inorganic salt is an optional but often desired ingredient to aid in composition thickening. Salts that may be used include NaCl, KCl, MgCl₂, CaCl₂), combinations thereof, or the like. Typically, the inorganic salt makes up 0 to 10% by weight, and preferably, 0.001 to 12% by weight, more preferably, from 0.05 to 4.5% by weight, and even more preferably, 0.09 to 2% by weight of the concentrated conditioning composition, based on the total weight of the concentrated conditioning composition, including any and all ranges and values subsumed therein.

Polymeric viscosity aids are an optional but often desired ingredient in the concentrated conditioning composition. Preferred polymers are those generally classified as high molecular weight ethoxylated fatty acid esters. Illustrative examples include PEG 120 methyl glucose dioleate, PEG 18 glyceryloleate/cocoate, PEG 150 pentaerythritol tetrastearate, or combinations thereof, or the like. One polymeric viscosity aid is PEG 150 pentaerythritol tetrastearate which is sold under the VERSATHIX™ name by Croda. When used, such aids make up from 0.001 to 0.8%, and preferably, from 0.01 to 0.5%, and most preferably, from 0.1 to 0.3% by weight of the total weight of the concentrated conditioning composition, including any and all ranges and values subsumed therein.

In some embodiments, less than 3.0% by weight sulfate is present in the concentrated conditioning composition, preferably less than 1.0% by weight, and most preferably, no (i.e., 0.0% by weight) sulfate based on the total weight of the concentrated conditioning composition, including any and all ranges and values subsumed therein.

As to anionic surfactants suitable for use in the concentrated conditioning composition, the anionic surfactant used can include aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate. The anionic surfactant can also be an alkyl sulfate (e.g., C₁₂-C₁₈ alkyl sulfate) or alkyl ether sulfate (including alkyl glyceryl ether sulfates). Among the alkyl ether sulfates are those having the formula:

RO(CH₂CH₂O)ₙSO₃M

wherein R is an alkyl or alkenyl having 8 to 18 carbons, preferably 12 to 18 carbons, n has an average value of at least 1.0, preferably less than 5, and most preferably 1 to 4, and M is a solubilizing cation such as sodium, potassium, ammonium or substituted ammonium.

The anionic may also include alkyl sulfosuccinates (including mono- and dialkyl, e.g., C₆-C₂₂ sulfosuccinates); alkyl and acyl taurates (often methyl taurates), alkyl and acyl sarcosinates, sulfoacetates, C₈-C₂₂ alkyl phosphates and phosphonates, alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Sulfosuccinates may be monoalkyl sulfosuccinates having the formula:

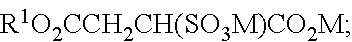

and amide-MEA sulfosuccinates of the formula:

R¹CONHCH₂CH₂O₂CCH₂CH(SO₃M)CO₂M wherein R¹ ranges from C₈-C₂₂ alkyl.

Sarcosinates are generally indicated by the formula:

R²CON(CH₃)CH₂CO₂M, wherein R² ranges from C₈-C₂₀ alkyl.

Taurates are generally identified by formula:

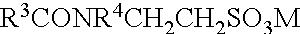

wherein R³ is a C₈-C₂₀ alkyl, R⁴ is a C₁-C₄ alkyl.

M is a solubilizing cation as previously described.

The isethionates that may be used include C₈-C₁₈ acyl isethionates (including those which have a substituted head group such as a C₁-4 alkyl substitution, preferably methyl substitution). These esters are prepared by a reaction between alkali metal isethionate with mixed aliphatic fatty acids having from 6 to 18 carbon atoms and an iodine value of less than 20. Often at least 75% of the mixed fatty acids have from 12 to 18 carbon atoms and up to 25% have from 6 to 10 carbon atoms.

The acyl isethionate used may be an alkoxylated isethionate such as is described in Ilardi et al., U.S. Pat. No. 5,393,466, entitled “Fatty Acid Esters of Polyalkoxylated isethonic acid; issued Feb. 28, 1995; hereby incorporated by reference in its entirety. This compound has the general formula:

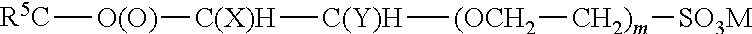

wherein R⁵ is an alkyl group having 8 to 18 carbons, m is an integer from 1 to 4, X and Y are each independently hydrogen or an alkyl group having 1 to 4 carbons and M is a solubilizing cation as previously described.

An anionic surfactant used can be sodium lauroyl glycinate, sodium cocoyl glycinate, sodium lauroyl glutamate, sodium cocoyl glutamate, sodium lauroyl isethionate, sodium cocoyl isethionate, sodium methyl lauroyl taurate, sodium methyl cocoyl taurate, or a combination thereof. Such anionic surfactants are commercially available from suppliers like Galaxy Surfactants, Clariant, Sino Lion and Innospec. Sodium cocoyl isethionate, sodium methyl lauroyl taurate, sodium lauroyl glyconate, sodium methyl lauroyl isethionate, or combinations thereof can be the preferred anionics suitable for use when used in the concentrated conditioning composition.

Amphoteric surfactants suitable (which depending on pH can be zwitterionic) include sodium acyl amphoacetates, sodium acyl amphopropionates, disodium acyl amphodiacetates and disodium acyl amphodipropionates where the acyl (i.e., alkanoyl group) can comprise a C₇-C₁₈ alkyl portion. Illustrative examples of the amphoteric surfactants include sodium lauroamphoacetate, sodium cocoamphoacetate, sodium lauroamphoacetate, sodium cocoamphoacetate and mixtures thereof.

As to the zwitterionic surfactants, such surfactants include at least one acid group. Such an acid group may be a carboxylic or a sulphonic acid group. They often include quaternary nitrogen, and therefore, can be quaternary amino acids. They should generally include an alkyl or alkenyl group of 7 to 18 carbon atoms generally comply with an overall structural formula:

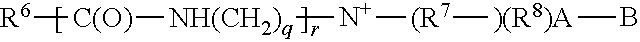

where R⁷ is alkyl or alkenyl of 7 to 18 carbon atoms; R⁷ and R⁸ are each independently alkyl, hydroxyalkyl or carboxyalkyl of 1 to 3 carbon atoms; q is 2 to 4; r is 0 to 1; A is alkylene of 1 to 3 carbon atoms optionally substituted with hydroxyl, and B is —CO₂— or —SO₃—.

Suitable zwitterionic surfactants for use in the present invention and within the above general formula include simple betaines of formula:

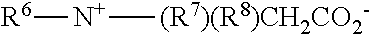

and amido betaines of formula:

R⁶—CONH(CH₂)ₜ—N⁺—(R⁷)(R⁸)CH₂CO₂⁻ where t is 2 or 3.

In both formulae R⁶, R⁷ and R⁸ are as defined previously. R⁶ may, in particular, be a mixture of C₁₂ and C₁₄ alkyl groups derived from coconut oil so that at least half, preferably at least three quarters of the groups Re have 10 to 14 carbon atoms. R⁷ and R⁸ are preferably methyl.

A further possibility is that the zwitterionic surfactant is a sulphobetaine of formula:

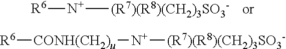

where u is 2 or 3, or variants of these in which —(CH₂)₃SO₃ is replaced by

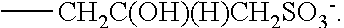

In these formulae, R⁶, R⁷ and R⁸ are as previously defined.

Illustrative examples of the zwitterionic surfactants include betaines like cocodimethyl carboxymethyl betaine, cocamidopropyl betaine, laurylamidopropyl betaine, or a combination thereof. An additional zwitterionic surfactant suitable for use includes cocamidopropyl hydroxysultaine, lauryl hydroxysultaine, or a combination thereof. Such surfactants are made commercially available from suppliers like Stepan Company, and it is within the scope of the concentrated conditioning composition to employ mixtures of the aforementioned surfactants. In an embodiment, the zwitterionic surfactant can be cocamidopropyl betaine.

Nonionic surfactants can optionally be used in the concentrated conditioning composition. When used, nonionic surfactants are typically used at levels as low as 0.5, 1, 1.5 or 2% by weight and at levels as high as 6, 8, 10 or 12% by weight of the total weight of the concentrated conditioning composition, including any and all ranges and values subsumed therein. The nonionics which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other nonionic surfactants include long chain tertiary amine oxides, long chain tertiary phosphine oxides, dialkyl sulphoxides, or a combination thereof.

Nonionic surfactants optionally used can include fatty acid/alcohol ethoxylates having the following structures a) HOCH₂(CH₂)ₛ(CH₂CH₂O)ᵥ H or b) HOOC(CH₂)_{c}(CH₂CH₂O)_{d} H; where s and v are each independently an integer up to 18; and c and d are each independently an integer from 1 or greater. In an embodiment of the invention, s and v are each independently 6 to 18; c and d are each independently 1 to 30. Other options for nonionic surfactants include those having the formula HOOC(CH₂)ᵢ—CH═CH—(CH₂)ₖ(CH₂CH₂O)_{z} H, where i, k are each independently 5 to 15; and z is 5 to 50. In another embodiment of the invention, i and k are each independently 6 to 12; and z is 15 to 35.

The nonionic surfactant can also include a sugar amide, such as a polysaccharide amide. Specifically, the nonionic surfactant may be one of the lactobionamides described in U.S. Pat. No. 5,389,279 to Au et al., entitled “Compositions Comprising Nonionic Glycolipid Surfactants issued Feb. 14, 1995; which is hereby incorporated by reference in its entirety, or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference in its entirety.

The pH of the concentrated conditioning composition can be 2 to 7, preferably, 3 to 6, and most preferably, 3.2 to 5.5, including any and all ranges and values subsumed therein. Adjusters to modify/buffer the pH may be used. Such pH adjusters include triethylamine, NaOH, KOH, H₂SO₄, HCl, C₆H₈O₇ (i.e., citric acid), C₃H₆O₃ (i.e., lactic acid), or a combination thereof. The pH adjusters are added at amounts to yield the desired final pH. The pH adjusters can include various alpha-hydroxy acids or a combination thereof. For example, the pH adjusters can comprise the alpha-hydroxy acids of citric acid, glycolic acid, lactic acid, malic acid, tartaric acid, or a combination thereof. The pH values can be assessed with commercial instrumentation such as a pH meter made commercially available from Thermo Scientific®. The pH adjusters can be present in an amount of 0.1 to 1.0% by weight of the total weight of the concentrated conditioning composition, including any and all ranges and values subsumed therein, for example, 0.25 to 0.75% by weight, for example, 0.5 to 0.6% by weight of the total weight of the concentrated conditioning composition, including any and all ranges and values subsumed therein.

Optional skin benefit agents suitable for use in the concentrated conditioning composition are limited only to the extent that they are capable of being topically applied.

Illustrative examples of the benefit agents suitable to include in the concentrated conditioning composition are acids, like amino acids, such as arginine, lysine, ceramide, valine, histidine, or a combination thereof. Additional water soluble benefit agents suitable for use include vitamin B₂, panthenol (vitamin B₅), niacinamide (vitamin B₃), vitamin B₆, vitamin C, biotin (vitamin B7), combinations thereof or the like. Water soluble derivatives of such vitamins may also be employed. For instance, vitamin C derivatives such as ascorbyl tetraisopalmitate, magnesium ascorbyl phosphate, or ascorbyl glycoside may be used alone or in combination with each other. Other water soluble benefit agents include 4-ethyl resorcinol, extracts like sage, aloe vera, green tea, grapeseed, thyme, chamomile, yarrow, cucumber, liquorice, rosemary leaf extract, Camellia sinensis leaf extract, pomegranate extract, ginger extract, collagen, hibiscus, or a combination thereof. Water soluble sunscreens like ensulizole can also be used. Total amount of optional water soluble benefit agents (including combinations) when present in the concentrated conditioning composition can be 0.0 to 10%, preferably 0.001 to 8%, and most preferably, 0.01 to 6% by weight, based on total weight of the concentrated conditioning composition and including any and all ranges subsumed therein.

It is also within the scope of the concentrated conditioning composition to optionally include oil (i.e., non-water) soluble benefit agents. In an embodiment, the concentrated conditioning composition can be substantially free of oil, meaning less than or equal to 0.01% by weight oil, based on the total weight of the concentrated cleansing composition. In an embodiment, the concentrated cleansing composition can comprise oils in an amount of less than or equal to 4.0% by weight oil, based on the total weight of the concentrated cleansing composition. For example, the oils can be present in an amount of 0.05% to 4.0% by weight oils. Illustrative examples of the oils that can be used include triglycerides (animal and/or vegetable) such as soybean oil, sunflower seed oil, coconut oil, palm kernel oil, castor oil, rapeseed oil, palm oil, grape seed oil, shea butter, cocoa butter, caprylic/capric triglyceride, safflower oil, fish oil, or a combination thereof.

Other oils desirable for use include mineral oil, jojoba oil, isoparaffins, a C₁₂-C₁₅ alkyl benzoates, polyalphaolefins, isohexadecane, silicone oils, siliconized waxes like siliconized beeswax, argan oil, marula oil, camellia oleifera seed oil, vegetable wax, petrolatum, a combination thereof (including with those oils above) or the like.

When petrolatum is used, the same typically has a melting point range from 25° to 65° C. Examples of such petrolatum are jellies like VASELINE® petroleum jelly made commercially available from Unilever, White Petroleum USP from Calumet Penreco as well as Petrolatum G2212 and White PROTOPET® from Sonneborn.

Thus, oil soluble actives or benefit agents are solubilized in the surfactants used. The only limitation with respect to such oil soluble benefit agents are that the same are suitable to provide a benefit when topically applied.

Illustrative examples of the types of oil soluble benefit agents that can optionally be used in the concentrated conditioning composition include components like stearic acid, vitamins like Vitamin A, D, E, and K (and their oil soluble derivatives), sunscreens like ethylhexylmethoxycinnamate, bis-ethyl hexyloxyphenol methoxyphenol triazine, 2-ethylhexyl-2-cyano-3,3-diphenyl-2-propanoic acid, drometrizole trisiloxane, 3,3,5-trimethyl cyclohexyl 2-hydroxybenzoate, 2-ethylhexyl-2-hydroxybenzoate, or a combination thereof.

Other optional oil soluble benefit agents suitable for use include resorcinols like 4-hexyl resorcinol, 4-phenylethyl resorcinol, 4-cyclopentyl resorcinol, 4-cyclohexyl resorcinol 4-isopropyl resorcinol, or a combination thereof. Also, 5-substituted resorcinols like 4-cyclohexyl-5-methylbenzene-1,3-diol, 4-isopropyl-5-methylbenzene-1,3-diol, or a combination thereof or the like may be used. The 5-substituted resorcinols, and their synthesis are described in commonly assigned U.S. Patent Publication No. 2016/0000669A1.

Even other oil soluble actives suitable for use include omega-3 fatty acids, omega-6 fatty acids, climbazole, farnesol, ursolic acid, myristic acid, geranyl geraniol, oleyl betaine, cocoyl hydroxyethyl imidazoline, hexanoyl sphingosine, 12-hydroxystearic acid, petroselinic acid, conjugated linoleic acid, terpineol, thymol, ProPlex (functional fatty acid), or a combination thereof.

The optional oil soluble benefit agent can be a retinoic acid precursor. The retinoic acid precursor can be retinol, retinal, retinyl propionate, retinyl palmitate, retinyl acetate, or a combination thereof. Retinyl propionate, retinyl palmitate, or a combination thereof can be typically preferred.

Still another retinoic acid precursor is hydroxyanasatil retinoate made commercially available under the name RETEXTRA® as supplied by Molecular Design International. The same may be used in a mixture with the oil soluble actives described herein.

When optional oil soluble active is used in the concentrated conditioning composition, it typically makes up from 0.0 to 1.5%, and preferably, from 0.001 to 1.5%, and most preferably, from 0.05 to 1.2% by weight of the total weight of the concentrated conditioning composition, including any and all ranges and values subsumed therein. In yet another embodiment, oil makes up from 0.1 to 0.5% by weight of the total weight of the concentrated conditioning composition, including any and all ranges and values subsumed therein.

Preservatives can desirably be incorporated into the concentrated conditioning composition to protect against the growth of potentially harmful microorganisms. Cosmetic chemists are familiar with appropriate preservatives and routinely choose them to satisfy the preservative challenge test and to provide product stability. Preservatives for use include hydantoin derivatives and propionate salts. Particularly preferred preservatives are sodium benzoate, benzoic acid, potassium sorbate, methylisothiazolinone (MIT), methylchloroisothiazolinone (CIT), iodopropynyl butyl carbamate, phenoxyethanol, 1,2-octanediol, hydroxyacetophenone, ethylhexylglycerine, hexylene glycol, methyl paraben, propyl paraben, imidazolidinyl urea, sodium dehydroacetate, dimethyl-dimethyl (DMDM) hydantoin, benzyl alcohol, or a combination thereof. Other preservatives include sodium dehydroacetate, chlorophenesin, decylene glycol, or a combination thereof. The preservatives should be selected having regard for the use of the composition and possible incompatibilities between the preservatives and other ingredients in the emulsion. Preservatives are preferably employed in amounts ranging from 0.01% to 2.0% by weight of the total weight of the concentrated conditioning composition (up to 7% by weight of total concentrated conditioning composition), including any and all ranges and value subsumed therein. Another preservative system can include hydroxyacetophenone alone or in a combination with other preservatives. Particularly preferred is a combination of CIT and MIT stabilized with magnesium nitrate. Such a preservative can be present in the concentrated conditioning composition in an amount of 0.01 to 1.0% by weight, for example, 0.03 to 0.9% by weight, for example, 0.6 to 0.08% by weight, including any and all ranges and values subsumed therein based on the total weight of the concentrated conditioning composition. The CIT: MIT preservative system can include as actives CIT and MIT, magnesium nitrate to stabilize, water, magnesium chloride, and acetic acid. Other preferred preservatives include sodium benzoate, benzoic acid, potassium sorbate, or a combination thereof.

Thickening agents are optionally suitable for use in the concentrated conditioning composition. Particularly useful are polysaccharides. Examples include fibers, starches, natural/synthetic gums, and cellulosics. Representative of the starches are chemically modified starches such as sodium hydroxypropyl starch phosphate, and aluminum starch octenylsuccinate. Tapioca starch is often preferred, as is maltodextrin. Suitable gums include xanthan, sclerotium, pectin, karaya, arabic, agar, guar (including Acacia senegal guar), carrageenan, alginate, or a combination thereof. Suitable cellulosics include hydroxypropyl cellulose, hydroxypropyl methylcellulose, ethylcellulose, sodium carboxy methylcellulose (cellulose gum/carboxymethyl cellulose), and cellulose (e.g., cellulose microfibrils, cellulose nanocrystals or microcrystalline cellulose). Sources of cellulose microfibrils include secondary cell wall materials (e.g., wood pulp, cotton), bacterial cellulose, and primary cell wall materials. Preferably the source of primary cell wall material is selected from parenchymal tissue from fruits, roots, bulbs, tubers, seeds, leaves, and combination thereof; more preferably is selected from citrus fruit, tomato fruit, peach fruit, pumpkin fruit, kiwi fruit, apple fruit, mango fruit, sugar beet, beet root, turnip, parsnip, maize, oat, wheat, peas, and combinations thereof; and even more preferably is selected from citrus fruit, tomato fruit, and combinations thereof. A most preferred source of primary cell wall material is parenchymal tissue from citrus fruit. Citrus fibers, such as those made available by HERBACEL® as AQ Plus can also be used as source for cellulose microfibrils. The cellulose sources can be surface modified by any of the known methods including those described in Colloidal Polymer Science, Kalia et al., “Nanofibrillated cellulose: surface modification and potential applications” (2014), Vol 292, Pages 5-31.

Synthetic polymers, in addition to polymeric viscosity aids, are yet another class of effective thickening agents that can optionally be used. This category includes crosslinked polyacrylates such as the Carbomers, polyacrylamides such as SEPIGEL® 305 and taurate copolymers such as SIMULGEL® EG and ARISTOFLEX® AVC, the copolymers being identified by respective INCI nomenclature as acrylate/sodium acryloyldimethyl taurate and acryloyl dimethyltaurate/vinyl pyrrolidone copolymer. Another preferred synthetic polymer suitable for thickening is an acrylate-based polymer made commercially available by Seppic and sold under the name SIMULGEL™ INS100. Calcium carbonate, fumed silica, and magnesium-aluminum-silicate can also be used.

The amounts of optional thickening agent, when used, may range from 0.001 to 5%, by weight of the compositions based on the total weight of the concentrated conditioning composition, including any and all ranges and values subsumed therein. Maltodextrin, xanthan gum, and carboxymethyl cellulose are the often preferred optional thickening agents. In an embodiment, the thickening agent can comprise comprises sodium chloride, silica, bentonite, magnesium aluminium silicate, carbomer, cellulose, or a combination thereof.

Fragrances, fixatives, opacifiers (like titanium dioxide or glycol distearate), and chelating agents may optionally be included in the antimicrobial composition. Possible chelating agents include, but are not limited to, ethylyene diaminetetraacetic acid (EDTA), diethylenetriaminepentaacetic acid (DTPA), ethylene diamine disuccinic acid (EDDS), pentasodium diethylenetriaminepentaacetate, trisodium N-(hydroxyethyl)-ethylenediaminetracetate, an acid form of EDTA, sodium thiocyanate, trisodium salt of methylglycinediacetic acid, tetrasodium glutamate diacetate and phytic acid, preferably wherein the chelating agent is ethylene diaminetetraacetic acid (EDTA), diethylenetriaminepentaacetic acid (DTPA), ethylene diamine disuccinic acid (EDDS), or a combination thereof. Each of these substances may be present in an amount of 0.03 to 5%, preferably 0.05 to 0.09% by weight of the total weight of the concentrated conditioning composition, including any and all ranges subsumed therein.

Emulsifiers having an HLB of greater than 8 may optionally be used. Illustrative examples include Tween, 40, 60, 80, polysorbate 20, or a combination thereof. Typically, emulsifiers for water continuous systems make up from 0.3 to 2.5% by weight of total weight of the concentrated conditioning composition, including any and all ranges and values subsumed therein.

Humectants can be employed as additives in the concentrated conditioning composition to assist in moisturization when such emulsions are topically applied. These are generally polyhydric alcohol type materials. Typical polyhydric alcohols include glycerol (i.e., glycerine or glycerin), propylene glycol, dipropylene glycol, polypropylene glycol (e.g., PPG-9), polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol, or a combination thereof. Most preferred is glycerin, propylene glycol, dipropylene glycol, or a combination thereof. In an embodiment, the humectant can be propylene glycol, butylene glycol, dipropylene glycol, glycerin, triethylene glycol, erythritol, capryl glycol, hyaluronic acid, polypropylene glycol-7 proypyl heptyl ether, or a combination thereof. The amount of humectant employed can be 0.0 to 35% by weight of the total weight of the compositions. Often, humectant makes up from 0.0 to 20%, and preferably, 0.001 to 15% by weight, more preferably, 0.5 to 1.0% by weight (most preferably, 0.75 to 12% by weight) of the total weight of the concentrated conditioning composition, including any and all ranges and values subsumed therein.

The concentrated conditioning composition can further comprise a hair repair agent. The hair repair agent can comprise glucono delta lactone, sodium gluconate, trehalose, sodium sulfate, or a combination thereof. The hair repair agent can be present in an amount of 0.3 to 1.5% by weight of the concentrated conditioning composition, for example, 0.4 to 1.2% by weight of the total weight of the concentrated conditioning composition, including any and all ranges and values subsumed therein.

The concentrated conditioning composition can additionally comprise polymers for volume. Such polymers include acrylate copolymers. For example, the acrylate copolymer can be present in an amount of 0.1 to 1.0% by weight, for example, 0.3 to 0.75% by weight, for example, 0.6% by weight of the total weight of the concentrated conditioning composition, including any and all ranges and values subsumed therein.

Color retention and frizz protection can be desired properties of the concentrated conditioning composition for the end user. Polymers can be useful at providing these desired properties. Such polymers can include acrylamidopropyltrimonium chloride/acrylamide copolymer, polyquaternium-7, or a combination thereof. Such a polymer can be present in an amount of 0.1 to 1.0% by weight of the concentrated conditioning composition, for example, 1.0% by weight of the total weight of the concentrated conditioning composition, including any and all values and ranges subsumed therein.

The concentrated conditioning composition has a viscosity of 200,000 to 700,000 centipoise (cP) (200 to 700 Pascal·seconds (Pa·s).

The concentrated conditioning composition has a level of concentration of 2.5, preferably the level of concentration is 2.0, as compared to a base, non-concentrated conditioning composition.

As used herein, “compositions” with no qualifier is meant to mean the concentrated conditioning composition. Hydratable, as used herein, means add and/or add and absorb water (i.e., to dilute) even to a composition that has water such as a composition that is initially 30 to 85% by weight water. Skin, as used herein, is meant to include skin on the arms (including underarms), face, feet, neck, chest, hands, legs, buttocks and scalp (including hair). Such end use composition is one suitable to be wiped or washed off, and preferably, washed off with water. The concentrated conditioning composition can be a home care cleaning composition but is preferably a shampoo, make-up wash, facial wash, hand wash or personal care liquid body wash. Viscosity, unless noted otherwise, is measured at 30° C. on a Brookfield DV2T using a Spindle T-Bar at 0.5 rpm for 60 seconds on a Helipath stand. The concentrated conditioning composition may, optionally, comprise medicinal or therapeutic agents, but preferably, is a wash which is cosmetic and non-therapeutic.

Except where otherwise explicitly indicated, all numbers in this description indicating amounts of material or conditions of reaction, physical properties of materials and/or use are to be understood as modified by the word “about.” All amounts are by weight of the final composition, unless otherwise specified.

It should be noted that in specifying any range of concentration or amount, any particular upper concentration can be associated with any particular lower concentration or amount as well as any subranges consumed therein. All ranges defined are meant to include all ranges subsumed therein. In that regard, it is noted that all ranges disclosed herein are inclusive of the endpoints, and the endpoints are independently combinable with each other (e.g., ranges of “up to 25% by weight, or, more specifically, 5% by weight to 20% by weight, in inclusive of the endpoints and all intermediate values of the ranges of 5% by weight to 25% by weight, etc.). “Combination is inclusive of blends, mixtures, alloys, reaction products, and the like. Furthermore, the terms “first”, “second”, and the like herein do not denote any order, quantity, or importance, but rather are used to distinguish one element from another. The terms “a” and “an” and “the” herein do not denote a limitation of quantity and are to be construed to cover both the singular and the plural, unless otherwise indicated herein or clearly contradicted by context. The suffix “(s)” as used herein is intended to include both the singular and the plural of the term it modifies, thereby including one or more of the term (e.g., the film(s) includes one or more films). Reference throughout the specification to “one embodiment”, “one aspect”, “another embodiment”, “another aspect”, “an embodiment”, “an aspect” and so forth means that a particular element (e.g., feature, structure, and/or characteristic) described in connection with the embodiment or aspect is included in at least one embodiment or aspect described herein and may or may not be present in other embodiments or aspects. In addition, it is to be understood that the described elements may be combined in any suitable manner in the various embodiments or aspects.

All cited patents, patent applications, and other references are incorporated herein by reference in their entirety. However, if a term in the present application contradicts or conflicts with a term in the incorporated reference, the term from the present application takes precedence over the conflicting term from the incorporated reference. While particular aspects have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents.

For the avoidance of doubt the word “comprising” is intended to mean “including” but not necessarily “consisting of” or “composed of.” In other words, the listed steps, options, or alternatives need not be exhaustive.

The disclosure of the invention as found herein is to be considered to cover all aspects as found in the claims as being multiply dependent upon each other irrespective of the fact that claims may be found without multiple dependency or redundancy. Unless otherwise specified, numerical ranges expressed in the format “from x to y” are understood to include x and y. In specifying any range of values or amounts, any particular upper value or amount can be associated with any particular lower value or amount. All percentages and ratios contained herein are calculated by weight unless otherwise indicated. The various features of the present invention referred to in individual sections above apply, as appropriate, to other sections mutatis mutandis. Consequently, features specified in one section may be combined with features specified in other sections as appropriate. Any section headings are added for convenience only and are not intended to limit the disclosure in any way.

### EXAMPLES

The following examples are merely illustrative of the concentrated cleansing compositions disclosed herein and are not intended to limit the scope hereof.

In the following examples, various formulations were made to determine levels of concentration for the conditioning compositions disclosed herein.

Table 1 includes a base formulation, which contains no concentration. Viscosity for this base formulation was 200,000 to 450,000 cp (200 to 450 Pa·s).

TABLE 1
Comparative Example 1: Base formulation (no concentration)

| | Inclusion of Ingredient in |
|---|---|
| Ingredient | Final formula |
| Water | Q.S |
| Cetearyl Alcohol | 4.00 |
| Dimethicone | 1.38 |
| Amodimethicone | 0.15 |
| Cetrimonium Chloride | 0.02 |
| Stearamidopropyl Dimethylamine | 1.00 |
| Behentrimonium Chloride | 0.70 |
| Dipropylene Glycol | 0.30 |
| Fragrance, preservatives, pH modifiers, | 2-4% |
| viscosity modifiers and | |
| additional performance ingredients | |

Table 2 includes a three-times (3×) concentrated conditioning composition. Viscosity of this 3× conditioning composition was not able to be measured because the composition was too thick and not processable.

TABLE 2
Comparative Example: 2 3x Concentrated
conditioning composition formulation

| | Inclusion of Ingredient in |
|---|---|
| Ingredient | Final formula |
| Water | QS |
| Cetearyl Alcohol | 12 |
| Dimethicone | 4.1409 |
| Amodimethicone | 0.4494 |
| Cetrimonium Chloride | 0.0642 |
| Stearamidopropyl Dimethylamine | 3 |
| Behentrimonium Chloride | 2.1 |
| Dipropylene Glycol | 0.9 |
| Lactic acid | 0.836 |
| Fragrance | 0.47 |
| Preservatives, pH modifiers, | 1-3% |
| viscosity modifiers and | |
| additional performance ingredients | |

The viscosity of this 3× conditioning composition was too high, resulting in a composition where the ingredients were not able to be incorporated homogeneously. Given the high level of solids in this composition and limited water available, the composition transformed into a thick, aerated solid that was not able to be processed after emulsification.

Table 3 includes a two and a half times (2.5×) concentrated conditioning composition. Viscosity of this 2.5× conditioning composition had values of 200,000 cP to 700,000 cP (200 to 700 Pa·s).

TABLE 3
Example 1: 2.5x Concentrated conditioning
composition formulation

| | Inclusion of Ingredient in |
|---|---|
| Ingredient | Final formula |
| Water | Q.S. |
| Cetearyl Alcohol | 8 |
| Dimethicone | 3.45075 |
| Amodimethicone | 0.3745 |
| Cetrimonium Chloride | 0.0535 |
| Stearamidopropyl Dimethylamine | 2.5 |
| Behentrimonium Chloride | 1.75 |
| Dipropylene Glycol | 0.75 |
| Lactic acid | 0.5808 |
| Fragrance | 0.47 |
| Preservatives, pH modifiers, | 1-3% |
| viscosity modifiers and | |
| additional performance ingredients | |

This 2.5× concentrated conditioning composition was able to be processed successfully and has a viscosity that allows it to be processed in large scale packages, has a flowable rheology, and can be to be packaged in the same type of containers used for non-concentrated compositions.

Table 4 includes a two-times (2×) concentrated conditioning composition. Viscosity of this 2× conditioning composition was 200,000 to 700,000 cP (200 to 700 Pa·s).

TABLE 4
Example 2: 2x Concentrated conditioning composition formulation

| | Inclusion of Ingredient in |
|---|---|
| Ingredient | Final formula |
| Water | Q.S. |
| Cetearyl Alcohol | 8 |
| Dimethicone | 2.7606 |
| Amodimethicone | 0.2996 |
| Cetrimonium Chloride | 0.0428 |
| Stearamidopropyl Dimethylamine | 2 |
| Behentrimonium Chloride | 1.4 |
| Dipropylene Glycol | 0.6 |
| Lactic acid | 0.5016 |
| Fragrance | 0.47 |
| Preservatives, pH modifiers, | 1-3% |
| viscosity modifiers and | |
| additional performance ingredients | |

This 2× concentrated conditioning composition was able to be processed successfully and has a viscosity that allows it to be processed in large scale packages, has a flowable rheology, and can be to be packaged in the same type of containers used for non-concentrated compositions.

1. A concentrated conditioning composition, comprising:
6 to 10% by weight of a C₆ to C₂₂ fatty alcohol;
1.0 to 10.0% by weight of a conditioning agent comprising a cationic surfactant, cationic polymers, or a combination thereof;
a humectant, wherein the humectant comprises propylene glycol, butylene glycol, dipropylene glycol, glycerin, triethylene glycol, erythritol, capryl glycol, hyaluronic acid, polypropylene glycol-7 propyl heptyl ether, or a combination thereof;
a thickening agent; and
60 to 90% by weight of water.

2. The concentrated conditioning composition of claim 1, wherein the fatty alcohol is a C₈ to C₂₀ alcohol.

3. The concentrated conditioning composition of claim 1, wherein the fatty alcohol comprises lauryl alcohol, myristyl alcohol, cetyl alcohol, stearyl alcohol, oleyl alcohol, or a combination thereof.

4. The concentrated conditioning composition of claim 1, wherein the conditioning agent comprises isohexadecane, a silicone, or a combination thereof, wherein the silicone conditioning agent comprises dimethicone, amodimethicone, cyclomethicone, dimethiconol and dimethiconol/silsesquioxane copolymer, or a combination thereof.

5. The concentrated conditioning composition of claim 1, wherein the cationic surfactant conditioning agent comprises behentrimonium chloride, stearamidopropyl dimethylamine, cetyl trimethyl ammonium chloride, stearyl trimethyl ammonium chloride, hydrogenated tallow alkyl trimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, N-(stearoyl colamino formyl methyl)pyridinium chloride, or a combination thereof.

6. The concentrated conditioning composition of claim 1, wherein the cationic polymer conditioning agent comprises copolymers of 1-vinyl-2-pyrrolidone and 1-vinyl-3-methylimidazolium salt; copolymers of 1-vinyl-2-pyrrolidone and dimethylaminoethyl methacrylate; cationic diallyl quaternary ammonium-containing polymers, or a combination thereof.

7. The concentrated conditioning composition of claim 1, further comprising an alpha-hydroxy acid.

8. The concentrated conditioning composition of claim 7, wherein the alpha-hydroxy acid comprises citric acid, glycolic acid, lactic acid, malic acid, tartaric acid, or a combination thereof.

9. The concentrated conditioning composition of claim 1, wherein the thickening agent comprises sodium chloride, silica, bentonite, magnesium aluminium silicate, carbomer, cellulose, or a combination thereof.

10. The concentrated conditioning composition of claim 1, further comprising a hair repair agent.

11. The concentrated conditioning composition of claim 10, wherein the hair repair agent comprises glucono delta lactone, sodium gluconate, trehalose, sodium sulfate, or a combination thereof.

12. The concentrated conditioning composition of claim 1, wherein the concentrated conditioning composition has a level of concentration of 2.5 as compared to a base, non-concentrated conditioning composition.

13. The concentrated conditioning composition of claim 2, wherein the fatty alcohol is a C₁₀ to C₁₈ alcohol.

14. The concentrated conditioning composition of claim 2, wherein the fatty alcohol is a C₁₆ to C₁₈ alcohol.

15. The concentrated conditioning composition of claim 12, wherein the level of concentration is 2.0.
